(19) United States
(12) Patent Application Publication
(10) Pub. No.: US 2003/0232018 A1
(43) Pub. Date: Dec. 18, 2003

(54) Stabilized formulations of adenovirus

(75) Inventors: Elisabeth Lehmberg, Kelseyville, CA (US); Erno Pungor, Millbrae, CA (US)

Correspondence Address: MILLEN, WHITE, ZELANO & BRANIGAN, P.C., 2200 CLARENDON BLVD., SUITE 1400, ARLINGTON, VA, 22201, US

(73) Assignees: BERLEX BIOSCIENCES, Richmond, CA; Schering AG, Berlin (DE)

(21) Appl. No.: 10/342,344

(22) Filed: Jan. 15, 2003

Related U.S. Application Data
(60) Provisional application No. 60/349,222, filed on Jan. 18, 2002.

(51) Int. Cl. A61K 39/12; A61L 9/04; A61K 31/70; A61K 31/08

(52) U.S. Cl. 424/45; 424/204.1; 514/25; 514/723; 514/718

(57) ABSTRACT

A method is disclosed to stabilize compositions comprising airborne viruses, particularly Adenoviruses; and more particularly recombinant Adenoviruses, by adding to the compositions a non-ionic detergent which comprises an alkyl moiety and a polyethylene glycol (PEG). Pharmaceutical and other compositions of Adenoviruses, particularly recombinant Adenoviruses suitable for methods of gene therapy, which comprise such detergents are also disclosed.

This application claims the benefit of the filing date of U.S. Provisional Application Serial No. 60/349,222 filed Jan. 18, 2002.

### DESCRIPTION OF THE INVENTION

This invention relates, e.g., to a method to stabilize a composition, such as pharmaceutical composition, which comprises a virus, such as an airborne virus (e.g., an Adenovirus), by adding to the composition a stabilizing-effective amount of a non-ionic detergent which comprises an alkyl moiety and, (a polyethylene glycol moiety [PEG (also known as a polyethyleneoxide structure), having the structure O—(CH₂CH₂O)_{z}—H, wherein Z is at least 2], e.g., a Brij detergent, or a polysorbate such as polysorbate 20. In a preferred embodiment, the non-ionic detergent has the structure shown in Formula I

R—O(CH₂CH₂O)_{X}—H I

wherein

X is 4-30, and

R is a linear or branched alklyl of 10-70 carbon atoms, optionally substituted by one or more (e.g., 1-3) carboxy, carbamide, halogen (F, Cl, Br, I), hydroxy, amino, or 1-3 rings, which can be aromatic (e.g., of 6-14 C atoms) or cycloalkyl (e.g., of 3-12 C atoms), which can also be heterocyclic (e.g., of 4-14 C atoms and 1-3 N, S, O or P atoms), and wherein said rings are optionally substituted by one or more alkyl (e.g., of 1-12 C atoms), hydroxy, amino, halogen (as above), nitro, sulfoxy, carboxy or carbamide (wherein ring groups can preferably be mono-, bi- or tricyclic),

or Formula II

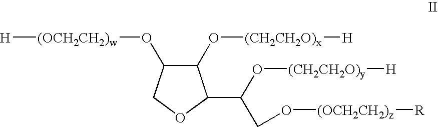

wherein R is —CO₂R having 10 to 70 carbons, and W+X+Y+Z=20, wherein R is as above for R,

or Formula III

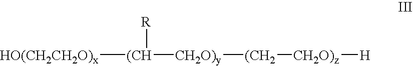

wherein R is as above, and X is 5-100, Y is 25-75 and Z is 50-100,

or Formula IV

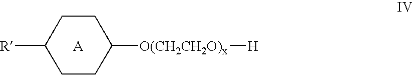

wherein X is 5-15, preferably 7-10, ring A is phenylene or cyclohexylene, and R′ is R as above, or combinations thereof.

In particularly preferred embodiments,

in Formula I, R is (CH₃(CH₂₎_{Y}—, wherein Y is 10-15; and in a most preferred embodiment, X is 23 and Y is 12 (Brij 35), or X is 20 and Y is 12 (Brij 58);

in Formula II, R is CₐH₍₂ₐ₊₁₎CO₂—, wherein a is 10 to 70, and in a most preferred embodiment, R═C₁₁H₂₃CO₂—(Tween 20);

in Formula III, R is CH₃, X is 55, Y is 29 and Z=55 (Pluronic F68), or R is CH₃, X is 98, Y is 67 and Z is 98 (Pluronic F-127); or

in Formula IV, R′ is (CH₃)₃C.CH₂C(CH₃)₂—, A is phenylene, and X is 9-10 (Triton X-100, NP40), or R′ is (CH₃)₃C.CH₂C(CH₃)₂—, A is cyclohexylene, and X is 9-10 (reduced Triton X-100).

One aspect of the invention is a method to stabilize a composition comprising an Adenovirus, comprising adding to the composition a stabilizing-effective amount of a non-ionic detergent of the invention, e.g., of Formulas I, II, III or IV as indicated above, or combinations thereof; wherein the Adenovirus is a recombinant Adenovirus which expresses a transgene, e.g. a therapeutic gene, for example for use in gene therapy; wherein the non-ionic detergent is a Brij detergent, such as Brij 35 or Brij 58, a polysorbate (Tween) detergent, such as polysorbate 20, 40, 60 or 65, particularly polysorbate 20, or a pluronic molecule, such as Pluronic F127 or F68, or a Triton-like molecule, such as Triton X-100, Triton X-114 or NP-40, in a concentration of about the critical micelle concentration (CMC), e.g., about 0.005% to about 0.1% (vol/vol), preferably about 0.05% to about 0.08%,more preferably about 0.05%.

Another aspect of the invention is a pharmaceutical composition comprising a stabilized Adenovirus composition prepared by the method described above and at least one pharmaceutically acceptable carrier.

Another aspect of the invention is a composition, e.g., a pharmaceutical composition, comprising an Adenovirus and a stabilizing-effective amount of non-ionic detergent of the invention, e.g., of Formulas I, II, III or IV as indicated above, or combinations thereof, and, optionally, one or more salts and/or excipients and in the case of a pharmaceutical composition, one or more pharmaceutically acceptable carriers, salts and/or excipients; wherein the Adenovirus is a recombinant Adenovirus which expresses a transgene, e.g., a therapeutic gene, for example one for use in gene therapy; wherein the neutral detergent is a Brij detergent, such as Brij 35 or Brij 58, a polysorbate (Tween) detergent, such as polysorbate 20, 40, 60 or 65, particularly polysorbate 20, a pluronic molecule, such as Pluronic F127 or F68, or a Triton-like molecule, such as Triton X-100, Triton X-114 or NP-40, in a concentration of about the CMC, e.g., about 0.005% to about 0.1% (vol/vol), preferably about 0.05% to about 0.08%, more preferably about 0.05%.

Another aspect is in a method of stabilizing a composition comprising Adenovirus, the improvement wherein a stabilizing-effective amount of a non-ionic detergent of the invention, e.g., of Formulas I, II, III or IV, is added to the composition.

Another aspect of the invention is a method of stabilizing a composition comprising an airborne virus, comprising adding to the composition a stabilizing-effective amount of a non-ionic detergent of the invention, e.g., of Formulas I, II, III or IV as indicated above, or combinations thereof. Another aspect of the invention is a composition, e.g., a pharmaceutical composition, comprising an airborne virus; a non-ionic detergent of the invention, e.g., of Formulas I, II, III or IV as indicated above, or combinations thereof; and, optionally, one or more salts or excipients. A pharmaceutical composition comprises one or more pharmaceutically acceptable carriers, salts and/or excipients.

By “stabilize”, e.g., stabilize a composition comprising a virus, is meant herein to inhibit a loss of available (measurable) amount and/or activity of the virus in the composition, over a defined period of time, compared to the amount of loss in a sample stored under the same conditions, but in the absence of the stabilizing agent.,

Typical degrees of stabilization achieved by the method of the invention are shown, e.g., in Example 2 and in FIGS. 1-6. For example, as shown in FIG. 1, highly purified Adenovirus compositions in a glass container, incubated at 2-8° C. in the absence of a stabilizer, lose about 2.5 logs (230 fold loss) of infectivity after one month; but when Tween 20 is present, the loss is less than about 0.5 log (about 3 fold loss). In other words, the recovered virus activity after 1 month at 2-8° C. in the presence of Tween 20 is approximately 80 times more than the recovered activity in the absence of Tween 20. FIG. 1 also shows that when the same experiment is carried out in plastic containers, the relative decrease in activity in the presence of Tween 20 is about 40%. In FIG. 2, viral concentration is measured (by HPLC). FIG. 2 shows, i.a., that, in either glass or plastic containers, Adenovirus compositions exhibit no detectable loss of Adenovirus concentration after one month at 2-8° C. in the presence of Tween 20. By contrast, the virus in a glass container loses about one third of its concentration after only 0.25 months at this temperature.

FIGS. 3 and 4 show that Tween 20 stabilizes Adenovirus compositions which are incubated at −70° C. FIG. 3 shows, i.a., that, in either glass or plastic containers, when the virus is incubated at −70° C., no detectable loss of infectivity occurs. The recovery of viral infectivity at any time between 1 and 12 months of incubation is about 0.5 to 0.8 logs higher(about 3 to 4 fold higher) in the presence of Tween 20 than in its absence. FIG. 4 shows similar findings when the concentration of virus is measured (by HPLC).

FIGS. 5 and 6 show that Tween 20 stabilizes Adenovirus compositions which are incubated at −20° C. FIG. 5 shows, i.a., that, in either glass or plastic containers, when the virus is incubated at −20° C., the recovery of viral infectivity remains substantially unchanged after 2.5 months of incubation when Tween 20 is present; but in the absence of Tween 20, infectivity decreases by about 0.6 logs (about 80%) after only 1 month of incubation. In FIG. 6, viral concentration is measured (by HPLC). FIG. 6 shows, i.a., that, in either glass or plastic containers, the concentration of virus remains substantially unchanged after as much as 14 months of incubation at −20° C. in the presence of Tween 20. When no Tween 20 is added, the concentration of virus drops below the limit of detectability in this assay. The recovery of virus is at least about 15 times better than in the absence of Tween 20 when incubated in either glass tubes or plastic tubes.

The invention relates to a method to stabilize a composition comprising a virus, e.g., an Adenovirus, comprising adding to the composition an amount of a non-ionic detergent as above, wherein the loss of virus amount and/or activity is less than about 30%, e.g., 0-30%, compared to the loss when said agent is not present, preferably less than about 10%, more preferably less than about 5% and most preferably less than about 2%, over a given period of time (e.g., at least 5 hours) at about 2-8° C., room temperature, 37° C., −20° C. or −70° C. Virus preparations can be stabilized to such degrees by the methods of the invention for at least about 5-24 hours, preferably for at least about 1-30 days, more preferably for at least about 1-12 months, and most preferably for at least about 2-3 years or longer. The amount of residual virus compared to the starting amount after a defined period of time can be greater than 2% up to, e.g., 100%), e g., greater than about 2%, 5%, 10%, 25% or 75%. In a most preferred embodiment, the amount is greater than about 90% (e.g., about 95, 98 or 99%).

By “activity” is meant herein the viability and/or infectivity (infectious units, infectious titer) of the virus.

Without wishing to be bound by theory, the stabilizers of the invention can function by, e.g. inhibiting self-aggregation of viruses and/or the binding (adsorption) of viruses to the surfaces of containers in which they reside, or to other components of the composition. Such stabilization is accomplished without interfering with the structural integrity of the viruses (e.g., the surface proteins are not denatured) or their infectivity.

In a preferred embodiment, an agent which stabilizes a composition of Adenovirus inhibits a loss in measurable Adenovirus concentration and/or activity which occurs during storage of the Adenovirus for a given period of time, at a particular temperature, compared to the decrease which occurs in the absence of the stabilizing agent.

One advantage of the inventive method is that it provides for stabilization of viruses at any of a variety of temperatures, for extended periods of time. This allows, for example, for long-term storage of viral preparations, particularly at temperatures above freezing, thereby eliminating the need for using costly refrigeration and/or freezer systems. The method is useful, e.g., for experimental purposes (e.g., for stabilizing Adenovirus preparations in glass or plastic autosampler vials prior to HPLC analysis); for the preparation, storage and/or preservation of pharmaceutical compositions; and for ensuring the preservation of the infectivity of viruses as reference agents, and in clinical specimens collected for diagnosis.

Any suitable detergent which is encompassed by the invention, e.g., by Formulas I, II, III or IV above, can be used in the methods or compositions of the invention. Formula I encompasses, for example, Brij 35 (when X is 23 and Y is 12); Brij 58 (when X is 20 and Y is 12); and Brij 3J (when X is 23 and Y is 11). Formula II encompasses, for example, a variety of polysorbates (polyoxyethylene 20 sorbitan molecules), including polysorbate 20 (polyoxyethylene 20 sorbitan monolaurate, Tween 20), polysorbate 40 (polyoxyethylene 20 sorbitan monopalmitate, Tween 40), polysorbate 60 (polyoxyethylene 20 sorbitan monostearate, Tween 60), and polysorbate 65. Formula III encompasses, for example, a variety of pluronics, including Pluronic 127 when X=98, Y=67 and Z=98, and Pluronic F68, when X=55, Y=29 and 7=55. Formula IV encompasses, for example, a variety of Triton-like molecules, for example, when X=9-10 and A=cyclohexylene, reduced Triton X-100, and when X=9-10 and A=phenylene, Triton X-100 or NP-40.

In a preferred embodiment, particularly for a composition which is a pharmaceutical composition, the detergent is one which has been approved for use in patients, e.g., an injectable grade detergent, such as injectable Tween-20.

Any suitable concentration of detergent can be used, provided that it is a stabilizing-effective amount, i.e., an amount which can achieve stabilization of the virus in a composition. Typically, the detergent is present at a concentration of about the CMC, e.g., about 0.005% to 0.1% vol/vol, preferably at about 0.05 to 0.08%, and more preferably at about 0.05%. Methods to determine how much detergent is required to stabilize the virus in a composition are conventional in the art. Typical methods to assay viral concentration or activity are described elsewhere herein.

Viruses which can be stabilized by the method of the invention will be evident to one of skill in the art. Such viruses can be pathogenic or non-pathogenic. In general, viruses that can be stabilized by the methods of the invention are airborne viruses. Among the preferred such viruses are, e.g., DNA or RNA viruses, such as viruses falling into the following families: Parvoviruses (including Adeno Associated Virus), Adenoviruses, Herpesviruses, Poxviruses, Hepatitis B-like Viruses, Picornoviruses, Calciviruses, Astroviruses, Togaviruses, Flaviviruses, Coronoviruses, Pararmyxoviruses, Rhabdoviruses, Filoviruses, Influenza viruses, Arenaviruses, Bunyaviruses, Reoviruses, Retroviruses, etc. Among viruses which can be stabilized by methods of the invention are viruses implicated in respiratory tract infections, such as, e.g., Rhinovirus, Parainfluenza virus and Respiratory Syncytial Virus (RSV). Viruses that can be stabilized by the methods of the invention include viruses with protein coats and hydrophobic surfaces.

Most preferred are Adenoviruses, e.g., avian or mammalian Adenoviruses, of any of the serotypes which have been identified, including Adenovirus 2 and Adenovirus 5. In a most preferred embodiment, recombinant viruses, such as, e.g. recombinant Adenoviruses which are suitable for gene therapy, are used. A variety of virus vectors have been described, including Adenoviruses defective in appropriate genes (e.g., E1 gene deficient Adenovirus), which are suitable for gene therapy applications. Any of a variety of genes can serve as, e.g., markers or as therapeutic agents, and can be cloned into such vectors under the control of suitable regulatory sequences and then introduced into patients in methods of, e.g., gene therapy. The selection of suitable vectors and genes which can be expressed therein, and methods to make such constructs and to use them for in vitro or ex vivo methods of gene therapy, are conventional and well-known to those of skill in the art (see, e.g., Sambrook, J. et al (1989). Molecular Cloning: A Laboratory Manual. Cold Spring Harbor Press, Cold Spring Harbor, N.Y.). Genes which can be used in the method of the invention include, e.g., genes encoding polypeptides such as enzymes, hormones, cytokines (e g., interferons or interleukins), growth factors (e.g., any of FGF-1 to FGF-23), etc. Also, marker genes such as, e.g., lacZ or Green Fluorescent Protein can be expressed. Of course, mutants or variant forms of any of the above viruses can be prepared (stabilized) by the method of the invention, as can recombinant, hybrid, chimeric, etc. forms of such viruses. Much of the discussion herein is directed to the preparation of Adenoviruses. However, one of skill in the art will recognize that any appropriate virus can be stabilized by the methods described herein, particularly airborne viruses. Methods of determining whether a particular virus can be stabilized by the detergents of the invention are conventional. Typical assays to measure viral concentration or activity are described elsewhere herein.

The term “stabilize an Adenovirus” as used herein refers to stabilizing a preparation comprising a single type of Adenovirus or multiple types, comprising a single Adenovirus particle or any number of particles.

Any suitable concentration of viruses can be stabilized by the method of the invention. For example, Adenoviruses can range from a concentration of about 1×10⁸ virus particles/mL to about 1×10¹³ virus particles/mL. Viruses having various degrees of purity can be stabilized by the method of the invention. For example, they can range from moderately purified preparations to highly purified preparations, such as viruses prepared by chromatography and membrane separation steps, e.g., ultrafiltration steps. The invention is particularly suitable for stabilization of highly purified virus preparations, e.g., near homogeneous preparations which are about 99.9% pure (e.g., which have less than 0.1% protein contamination). Unless otherwise stabilized, such highly purified preparations rapidly lose infectivity during storage. For example, the recovery of highly-purified Adenoviruses from glass autosampler vials (e.g., RP-HPLC or SEC-HPLC) has been observed to be only about 71% after 16 hours of storage at room temperature, and only about 60% after 2 hours, as measured by HPLC analysis. The loss is believed to be due, at least in part, to adsorption of viral particles to the walls of the autosampler vials, without wishing to be bound by theory.

Viruses can be stabilized by any of a variety of regimens. For example, a detergent of the invention can be added to a liquid preparation of Adenovirus; or it can be added to a container of frozen Adenovirus, either before, during or after thawing; or it can be added to a liquid preparation which is then lyophilized.

Methods to measure the amount (mass, concentration) of viruses are routine and conventional. For Adenoviruses, for example, one can measure the amount of viral particles by, e.g., HPLC, (e.g., by determining the amount of a capsid protein, such as hexon), or can determine the number of viral particles by, e.g., Particle Count Determination. Such measurements detect the amount of available (measurable) viral mass, e.g., the amount of virus which is not adsorbed to the walls of the container in which it resides. See, e.g., Example 2, which illustrates the use of RP-HPLC to measure virus concentration. By determining and comparing the amount of two or more different capsid components, one can also determine whether the virions are intact. Measurement with HPLC may allow one to detect changes (e.g., oxidations, deamidations, etc.) in coat protein molecules, which can affect, e.g. immunogenicity, biodistribution, etc. of the virus.

Methods to measure the activity (e.g., viability and/or infectivity) of viruses are also routine and conventionial. For Adenoviruses, for example, one can measure the number of infectious particles with, e.g., cytopathic effect (CPE), end point dilution (EPD), or a plaque forming assay, or can use FACS analysis, e.g., in conjunction with FITC labeled anti-penton (coat protein) antibody. See, e.g., Mittereder et al. (1996). J. Virology 70, 7498. Such measurements detect the amount of available (measurable) viral infectivity, e.g., infective virions that are not adsorbed to other virions or to the walls of the container in which they reside. See, e.g., Example 2, which illustrates the use of endpoint dilution to measure viral infectivity. In the case of a recombinant Adenovirus which expresses a transgene, activity of the Adenovirus correlates with the amount of expression of the transgene; thus, activity can be measured by quantifying the amount or activity of transgene expressed.

One can measure viral concentration or activity at any of a variety of time points. For example, after transferring a virus composition into a container, there may be an initial rapid loss of viral concentration or activity (possibly as a result of virus adhering to container walls), followed by a period of slower loss. One of skill in the art can choose appropriate time periods during which to perform the assays, depending on the variables being studied.

The invention also contemplates pharmaceutical compositions which comprise an effective amount of a virus, such as an Adenovirus. By “effective amount” is meant herein an amount which is effective for achieving a therapeutic effect. For example, an effective amount of a recombinant Adenovirus comprising a CF gene is one which, when administered to a cystic fibrosis patient, is effective to reduce the symptoms of the disease.

Pharmaceutical compositions of the invention contain any of a variety of conventional pharmaceutically acceptable carriers. In a preferred embodiment, the pharmaceutical compositions are in liquid form, although they can also be in solid (e.g., lyophilized) form. A pharmaceutical composition of the invention comprises sterile water (e.g., USP grade water for injection) and, optionally, a conventional buffer, such as, e.g., PBS, at a pH ˜6.5 to 7.5, preferably about 7, and at a concentration of about 0.1X to 4X or Tris, at a pH ˜7 to 8, preferably about 7.5, and at a concentration of about 0.05M to 0.1M. Other buffers which are effective at neutral pH, such as citrate buffer, can also be used. A composition of the invention can also comprise, optionally, salts (e.g., MgCl₂, at a concentration of about 1-5 mM, preferably about 2 mM), and/or agents to modulate osmolarity/osmolality, such as, e.g., sucrose, at a concentration of about 1-8%, preferably about 2% (±10%) (wt/vol).

In a most preferred embodiment, a pharmaceutical composition of the invention comprises about 5×10⁷ to 5×10¹¹ particles/mL of Adenovirus Tween 20 at about 0.05% (vol/vol), about 2 mM MgCl₂ and about 2% (wt/vol) sucrose, in 1X PBS, at a pH of about 6.95. Optionally, particularly when in the form of a pharmaceutical composition, a composition of the invention can contain one or more other conventional pharmaceutically acceptable excipients or stabilizers.

Formulations of the invention are stable when in any of a variety of containers, e.g., glass or plastic containers, such as vials or syringes (e.g., Hypak syringes which comprise interior silicone coatings), made of any of a variety of plastic materials, such as polypropylene, polyethylene or polycarbonate, or glass, such as brown or white borosilicate HPLC vials.

Pharmaceutical compositions of the invention can be used in a variety of therapeutic applications. For example, a recombinant Adenovirus which expresses a therapeutic transgene can be used in methods of gene therapy, in which the transgene substitutes for a defective gene, provides an enhanced immunological response, or the like.

### BRIEF DESCRIPTION OF THE DRAWINGS

FIG. 1 illustrates that Tween 20 stabilizes Adenovirus compositions incubated at 2-8° C., in either glass or plastic containers, as measured by infectivity assays.

FIG. 2 illustrates that Tween 20 stabilizes Adenovirus compositions incubated at 2-8° C. in either glass or plastic containers, as measured by HPLC.

FIG. 3 illustrates that Tween 20 stabilizes Adenovirus compositions incubated at −70° C., in either glass or plastic containers, as measured by infectivity assays.

FIG. 4 illustrates that Tween 20 stabilizes Adenovirus compositions incubated at −70° C., in either glass or plastic containers, as measured by HPLC.

FIG. 5.illustrates that Tween 20 stabilizes Adenovirus compositions incubated at −20° C., in either glass or plastic containers, as measured by infectivity assays.

FIG. 6 illustrates that Tween 20 stabilizes Adenovirus compositions incubated at −20° C., in either glass or plastic containers, as measured by HPLC.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

### Example 1

### Testing Various Agents for their Ability to Stabilize Adenovirus Compositions

Adenoviruses, e.g., Adenovirus type 5, disintegrate into their proteins upon injection onto a RP-HPLC column and the proteins separate when eluted with an acetonitrile/TFA gradient. A characteristic protein fingerprint is obtained which is useful for quantification and purity analysis of some steps in an Adenovirus production protocol. However, unless otherwise stabilized, it has been observed that the yields of viral protein peaks are variable and decrease with time as samples are held in the autosampler tubes.

To investigate this phenomenon, highly purified Adenovirus samples are stored for several hours in an autosampler system prior to being injected into the HPLC column. Unless otherwise stabilized, the integrated areas of the viral protein peaks, (e.g., under the hexon peak) as measured by a conventional RP-HPLC or SEC-HPLC assay, decrease over time when the samples are stored for several hours, at either room temperature or at 4° C., in the autosampler system. For example, recovery is only about 70% after 1.6 hours storage at room temperature, and only about 60% after two hours. The losses are shown to be due, at least in part, to binding of the viruses to surfaces (walls of the autosampler tubes); this binding is sometimes mediated by precipitation of virus aggregates (virus binding to other viruses).

Several agents are added in an effort to counteract these adsorptive processes, and are tested for their ability to stabilize the compositions. The samples are incubated in autosampler tubes for any desired time, e.g., for about 1-20 hours. If desired, assays are performed at desired time points, e.g., at equally spaced time points during the course of the assay. Plurmic, Brij 58 and Tween 20 are among the agents tested.

Further experiments are carried out with Brij 58 and Tween 20 to determine the effects of, e g., varying the types of HPLC storage vials, temperature of storage, and the concentration of the detergent (ranging from 0 to 0.1%, vol/vol). The effect of freezing virus samples in the presence of detergent is also evaluated. The optimal concentrations of Brij 58 and Tween 20 are each about 0.05% (vol/vol). At 0.1%, both detergents appear to result in partial disruption of the virus. Tween 20 allows for freezing of the virus and better quantitative recovery from HPLC vials than does Brij 58. The addition of 0.05% Tween 20 provides more than 16 hours of stability in the autosampler after thawing the virus sample.

### Example 2

### Testing Tween 20 Under Various Conditions

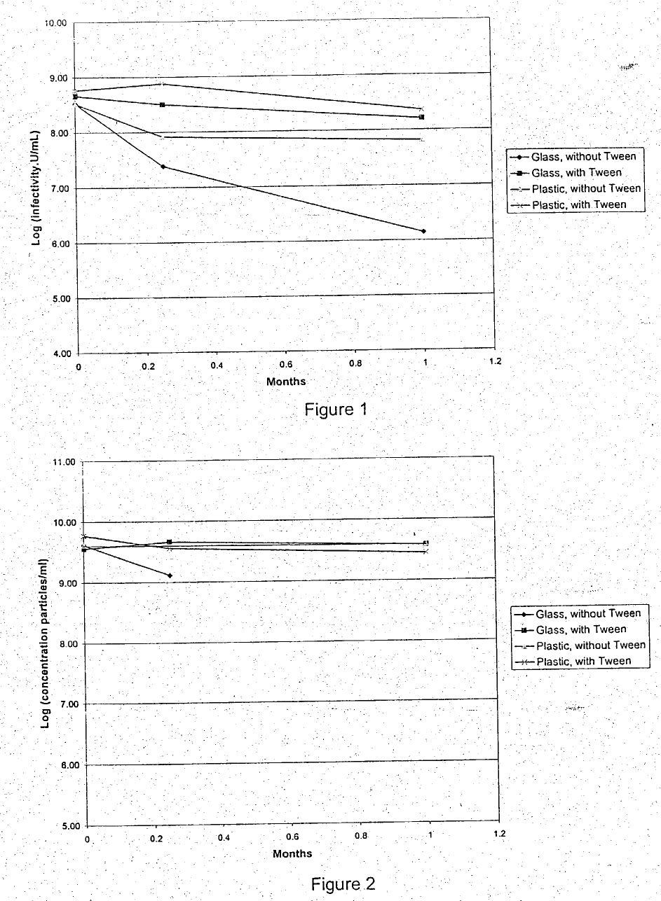

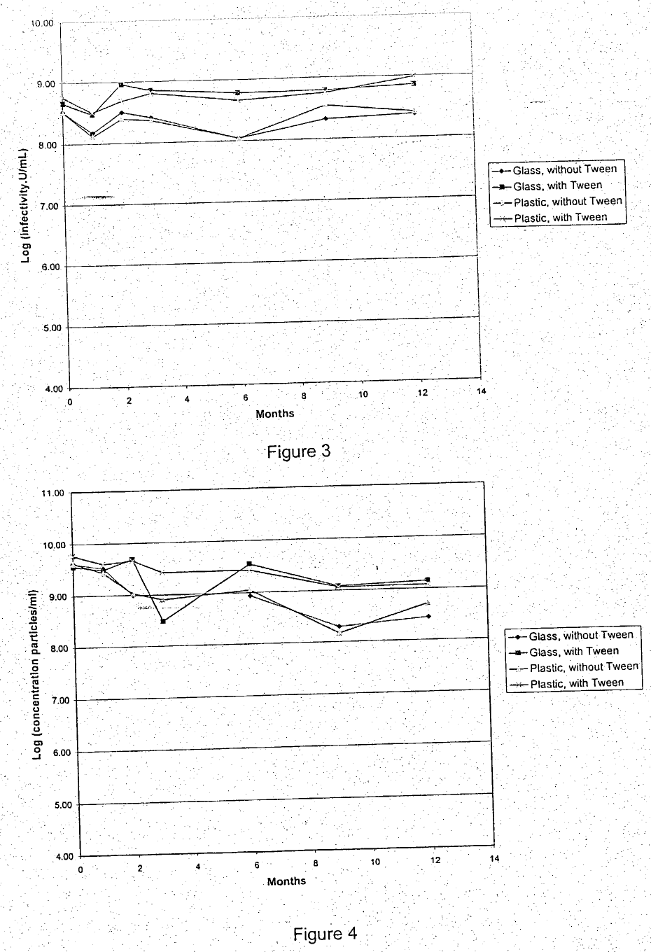

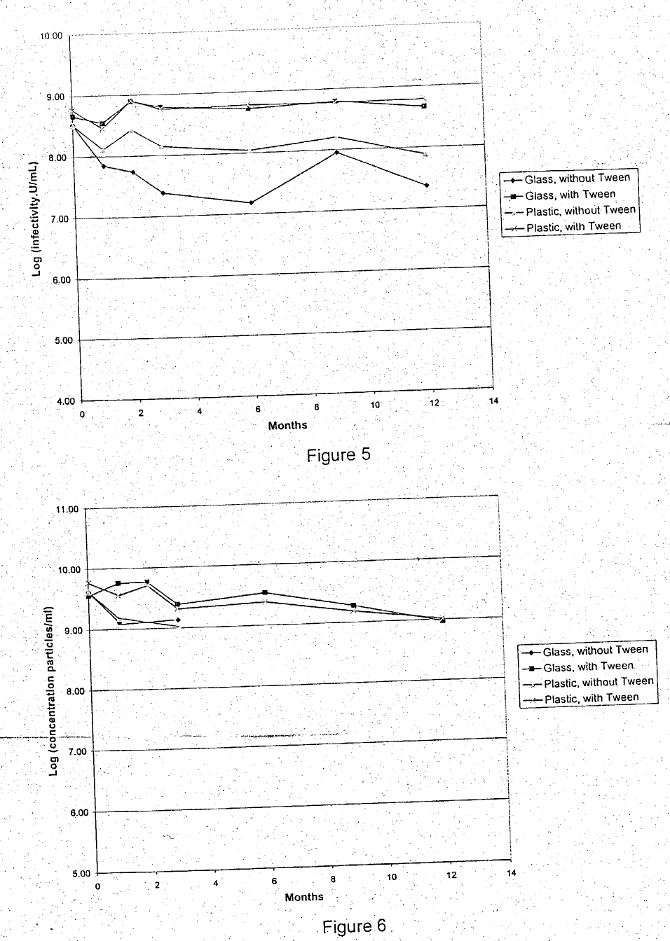

Adenovirus compositions are incubated at 2-8° C., −20° C. or −70° C., in glass or plastic containers (e.g., vials or syringes), for up to 1 month (2-8° C.) or 14 months (−20° C. and −70° C.). Aliquots are assayed periodically, either by end point dilution or by RP HPLC analysis. The results of typical experiments are shown in FIGS. 1-6 and discussed elsewhere herein. Under all conditions tested, higher stability of the virus is obtained when the samples are incubated in the presence of Tween 20 than in the absence of Tween 20.

### Example 3

### Testing Tween 20 Under Still Other Conditions

Tests are performed as described in Example 2, but additional parameters, such as the optimal concentration of Tween 20, incubation at room temperature (about 20-25° C.) and 37° C., and longer times of incubation (e.g., up to about 2-3 or more years) are also tested. The results confirm that at room temperature, and at longer periods of incubation, Tween 20 effectively stabilizes Adenovirus compositions.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications cited above are hereby incorporated by reference.

Aspects of the invention include:

A method to stabilize a composition comprising Adenovirus, comprising adding to the composition a stabilizing-effective amount of a non-ionic detergent which comprises an alkyl moiety and a polyethylene glycol (PEG) moiety;

A method to stabilize a composition comprising Adenovirus, comprising adding to the composition a stabilizing-effective amount of a non-ionic detergent of Formula I

R—O(CH₂CH₂O)_{X}—H I

wherein X is 4-30, and

R is a linear or branched alkyl of 10-70 carbon atoms, optionally substituted by one or more of carboxy, carbamide, halogen, hydroxy or amine, or by 1-3 rings, which can be aromatic or cycloalkyl, and can also be heterocyclic, and which can optionally be substituted by one or more alkyl, hydroxy, amine, halogen, nitro, sulfoxy, carboxy or carbamide, groups,

or Formula II

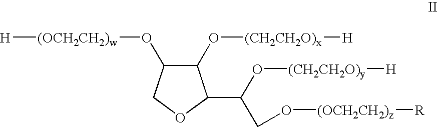

wherein R is a —CO₂R′ having 10 to 70 carbons, and W+X+Y+Z=20, wherein R′ is as defined above for R,

or Formula III

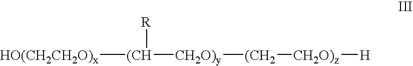

wherein R is as above, and X is 5-100, Y is 25-75 and Z is 50-100,

or Formula IV

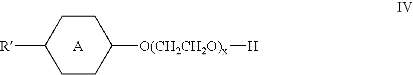

wherein X is 5-15, ring A is phenylene or cyclohexylene, and R′ is R as above, or combinations thereof,

A method to stabilize a composition comprising Adenovirus, comprising adding to the composition a stabilizing-effective amount of a non-ionic detergent of Formula I

R—O(CH₂CH₂O)_{X}—H II

wherein X is 4-30, and R is (CH₃)(CH₂)_{Y}—, wherein Y is 10-15,

or Formula II

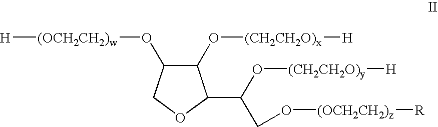

wherein R is CₐH₍₂ₐ₊₁₎CO₂—, wherein a is 10 to 70,

or Formula III

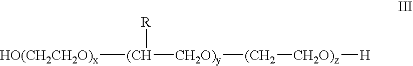

wherein R is CH₃, X is 55, Y is 29 and Z is 55 (Pluronic F68), or R is CH₃ X is 98, Y is 67 and Z is 98 (Pluronic F127),

or Formula IV,

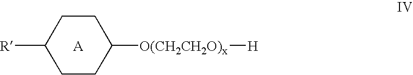

wherein R′ is (CH₃)₃C.CH₂C(CH₃)₂—, A is phenylene, and X is 9-10 (Triton X-100, NP40), or R′ (CH₃)₃C.CH₂C(CH₃)₂—, A is cyclohexylene, and X is 9-10 (reduced Triton X-100),

or combinations thereof,

A method to stabilize a composition comprising Adenovirus, comprising adding to the composition a stabilizing-effective amount of a non-ionic detergent of Formula I

R—O(CH₂CH₂O)_{X}—H I

wherein R is (CH₃)(CH₂)_{Y}, and

wherein X is 23 and Y is 12 (Brij 35), or X, is 20 and Y is 12 (Brij 58),

or Formula II

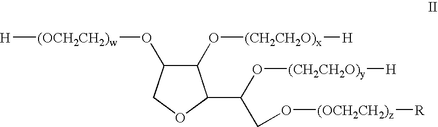

wherein R is R═C₁₁H₂₃CO₂—(Tween 20),

or combinations thereof;

A method as above, wherein the detergent is polysorbate 20 (Tween 20), wherein the Adenovirus is a recombinant Adenovirus, where in the Adenovirus is a recombinant Adenovirus suitable for gene therapy, wherein the detergent is in a concentration of 0.005% to 0.08% (vol/vol), wherein the detergent is in a concentration of 0.05 to 0.08% (vol/vol), or wherein the detergent is in a concentration of 0.05% (vol/vol);

A pharmaceutical composition, comprising a stabilized Adenovirus composition made by a method of the invention and at least one pharmaceutically acceptable carrier;

A pharmaceutical composition, comprising

a) an Adenovirus,

b) a stabilizing-effective amount of a non-ionic detergent according to the invention; and

c) at least one pharmaceutically acceptable carrier;

wherein the Adenovirus is a recombinant Adenovirus, wherein the Adenovirus is a recombinant Adenovirus suitable for gene therapy, wherein the detergent is in a concentration of 0.005%to 0.1% (vol/vol), or 0.05 to 0.08% (vol/vol), or 0.05% (vol/vol);

A pharmaceutical composition of the invention, wherein the Adenovirus is a recombinant Adenovirus suitable for gene therapy, the detergent is Tween 20 at a concentration of 0.05% (vol/vol), and the pharmaceutically acceptable carrier comprises 2 mM MgCl₂, and 2% sucrose (wt/vol), and, preferably, sterile water.

Other aspects include:

A method to stabilize a composition comprising an airborne virus, comprising adding to the composition a stabilizing-effective amount of a non-ionic detergent which comprises an alkyl moiety and a polyethylene glycol (PEG) moiety;

A method to stabilize a composition comprising an airborne virus, comprising adding to the composition a stabilizing-effective amount of a non-ionic detergent of Formula I

R—O(CH₂CH₂O)_{X}—H I

wherein X is 4-30, and

R is a linear or branched alkyl of 10-70 carbon atoms, optionally substituted by one or more of carboxy, carbamide, halogen, hydroxy or amine, or by 1-3 rings, which can be aromatic or cycloalkyl, and can also be heterocyclic, and which can optionally be substituted by one or more alkyl, hydroxy, amine, halogen, nitro, sulfoxy, carboxy or carbamide groups,

or Formula II

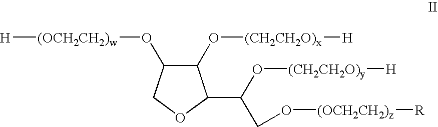

wherein R is —CO₂R′ having 10 to 70 carbons, and W+X+Y+Z=20, wherein R′ is as defined above for R,

or Formula III

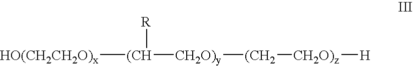

wherein R is as above, and X is 5-100, Y is 25-75 and Z is 50-100,

or Formula IV

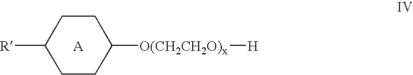

wherein X is 5-15, ring A is phenylene or cyclohexylene, and R′ is R as above, or combinations thereof,

A method to stabilize a composition comprising an airborne virus, comprising adding to the composition a stabilizing-effective amount of a non-ionic detergent of Formula I

R—O(CH₂CH₂O)_{X}—H I

wherein X is 4-30, and R is (CH₃)(CH₂)_{Y}—, wherein Y is 10-15

or Formula II

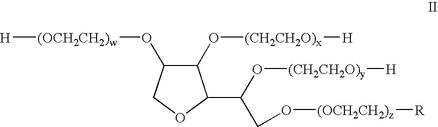

wherein R is CₐH₍₂ₐ₊₁₎CO₂—, wherein a is 10 to 70

or Formula III

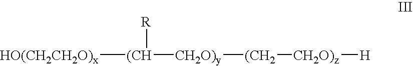

wherein R is CH₃,X is 55, Y is 29 and Z is 55 (Pluronic F68), or R is CH₃,X is 98, Y is 67 and Z is 0.98 (Pluronic F127),

or Formula IV,

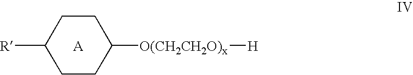

wherein R′ is (CH₃)₃C.CH₂C(CH₃)₂—, A is phenylene, and X is 9-10(Triton X-100, NP40), or R′ (CH₃)₃C.CH₂C(CH₃)₂—, A is cyclohexylene, and X is 9-10 (reduced Triton X-100), or combinations thereof,

A method to stabilize a composition comprising an airborne virus, comprising adding to the composition a stabilizing-effective amount of a non-ionic detergent of Formula I

R—O(CH₂CH₂O)_{X}—H I

wherein R is (CH₃)(CH₂)_{Y}—, and

wherein X is 23 and Y is 12 (Brij 35), or X is 20 and Y is 12 (Brij 58),

or Formula II

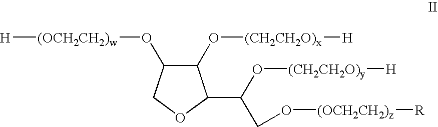

wherein R is R=C₁₁H₂₃CO₂—(Tween 20),

or combinations thereof,

A method as above, wherein the detergent is polysorbate 20 (Tween 20), of 0.005% to 0.1% (vol/vol);

A pharmaceutical composition comprising a stabilized airborne virus made by a method of the invention, and at least one pharmaceutically acceptable carrier;

A pharmaceutical composition comprising an airborne virus, comprising

a) said virus,

b) a stabilizing-effective amount of a non-ionic detergent according to the invention, and

c) at least one pharmaceutically acceptable carrier;

wherein the detergent is in a concentration of 0.005% to 0.1% (vol/vol).

Another aspect of the invention is a method of stabilizing a composition comprising Adenovirus, the improvement wherein a stabilizing-effective amount of a non-ionic detergent of the invention is added to the composition;

A composition comprising an airborne virus preferably, adenovirus, and a stabilizing-effective amount of a non-Ionic detergent which comprises an alkyl moiety and a polyethylene glycol (PEG) moiety; in other preferred aspects such a composition comprises non-ionic detergents of Formulae I-IV or other sub aspects as described above for the other compositions of this invention.

The entire disclosure of all applications, patents and publications, cited herein and of U.S. Provisional Application Serial No. 60/349,222, filed Jan. 18, 2002 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential various changes and modifications of the invention to adapt it to various usages and conditions.

1. A method for stabilizing a composition comprising an airborne virus comprising adding to the composition a stabilizing-effective amount of a non-ionic detergent of Formula I
R—O(CH₂CH₂O)_{X}—H I
wherein
X is 4-30, and R is a linear or branched alkyl of 10-70 carbon atoms,
or Formula II 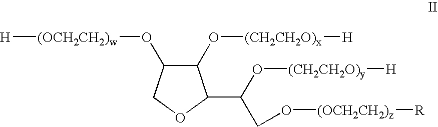
wherein R is —CO₂R′ having 10 to 70 carbons, and W+X+Y+Z=20, wherein R′ is as above for R,
or Formula III 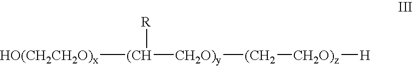
wherein R is as above, and X is 5-100, Y is 25-75 and Z is 50-100,
or Formula IV 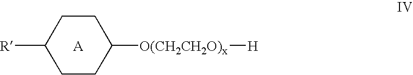
wherein X is 5-15 ring A is phenylene or cyclohexylene, and R′ is R as above,
or a combination thereof.

2. The method of claim 1 wherein said airborne virus is an Adenovirus.

3. The method of claim 2 wherein the Adenovirus is a recombinant Adenovirus expressing a transgene.

4. The method of claim 1, wherein the non-ionic detergent is a Brij detergent, a polysorbate (Tween) detergent, a pluronic molecule, or a Triton-like molecule.

5. The method of claim 1, wherein the concentration ranges from about 0.005% to about 0.1% (vol/vol).

6. The method of claim 5, wherein the concentration ranges from about 0.05% to about 0.08% (vol/vol).

7. A method for stabilizing a composition comprising an airborne virus comprising adding to the composition a stabilizing-effective amount of a non-ionic detergent which comprises an alkyl moiety and a polyethylene glycol moiety.

8. The method of claim 7 wherein said airborne virus is an Adenovirus.

9. The method of claim 7, wherein said non-ionic detergent has the structure shown in Formula I
R—O(CH₂CH₂O)_{X}—H I
wherein
X is 4-30, and R is a linear or branched alkyl of 10-70 carbon atoms,
or Formula II 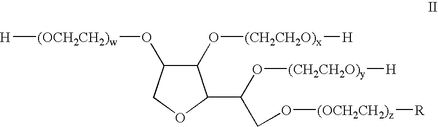
wherein R is —CO₂R′ having 10 to 70 carbons, and W+X+Y+Z=20, wherein R′ is as above for R, or Formula III 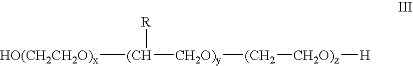
wherein R is as above, and X is 5-100, Y is 25-75 and Z is 50-100,
or Formula IV 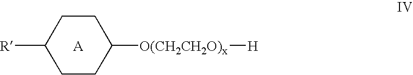
wherein X is 5-15 ring A is phenylene or cyclohexylene, and R′ is R as above,
or a combination thereof.

10. The method of claim 9, wherein R is substituted by one or more of carboxy, carbamide, halogen, hydroxy, amino, or 1-3 rings, which can be aromatic or cycloalkyl or can also be heterocyclic.

11. The method of claim 10, wherein said 1-3 rings are substituted by one or more of alkyl, hydroxy, amino, halogen, nitro, sulfoxy, carboxy or carbamide.

12. The method of claim 9 wherein,
in Formula I, R is (CH₃)(CH₂)_{Y}—, wherein Y is 10-15;
in Formula II, R is CₐH₍₂ₐ₊₁₎CO₂—, wherein a is 10 to 70;
in Formula III, R is CH₃, X is 55,Y is 29 and Z=55,or R is CH₃,X is 98,Y is 67 and Z is 98; or
in Formula IV, R′ is (CH₃)₃C.CH₂C(CH₃)₂—, A is phenylene, and X is 9-10, or R′ is (CH₃)₃C.CH₂C(CH₃)₂—, A is cyclohexylene, and X is 9-10.

13. The method of claim 12 wherein in Formula I R is (CH₃)(CH₂)_{Y}—, X is 23 and Y is 12 or R is (CH₃)(CH₂)_{Y}—, X is 20 and Y is 12.

14. The method of claim 12 wherein in Formula II R is C₁₁H₂₃CO₂—.

15. A method for stabilizing a composition comprising an Adenovirus comprising adding to the composition a stabilizing-effective amount of a non-ionic detergent which comprises an alkyl moiety and a polyethylene glycol (PEG) moiety.

16. A method for stabilizing a composition comprising an Adenovirus, comprising adding to the composition a stabilizing-effective amount of a non-ionic detergent of Formula I
R—O(CH₂CH₂O)_{X}—H I
wherein X is 4-30, and
R is a linear or branched alkyl of 10-70 carbon atoms,
or Formula II 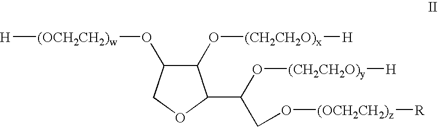
wherein R is a —CO₂R′ having 10 to 70 carbons, and W+X+Y+Z=20, wherein R′ is as defined above for R,
or Formula III 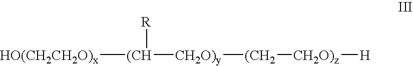
wherein R is as above, and X is 5-100, Y is 25-75 and Z is 50-100,
or Formula IV 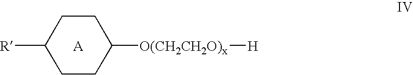
wherein X is 5-15, ring A is phenylene or cyclohexylene, and R′ is R as above,
or a combination thereof.

17. The method of claim 16, wherein said alkyl is substituted by one or more of carboxy, carbamide, halogen, hydroxy or amine, or by 1-3 rings, which can be aromatic or cycloalkyl or can also be heterocyclic.

18. The method of claim 17, wherein said 1-3 rings can be substituted by one or more of alkyl, hydroxy, amine, halogen, nitro, sulfoxy, carboxy or carbamide.

19. A method for stabilizing a composition comprising Adenovirus, comprising adding to the composition a stabilizing-effective amount of a non-ionic detergent of Formula I
R—O(CH₂CH₂O)_{X}—H I
wherein X is 4-30, and R is (CH₃)(CH₂)_{Y}—, wherein Y is 10-15,
or Formula II 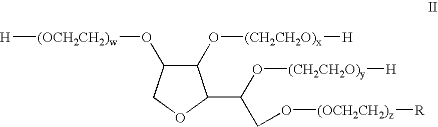
wherein R is CₐH₍₂ₐ₊₁₎CO₂—, a is 10 to 70,
or Formula III 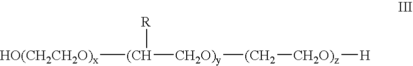
wherein R is CH₃, X is 55, Y is 29 and Z is 55, or R is CH₃, X is 98, Y is 67 and Z is 98,
or Formula IV, 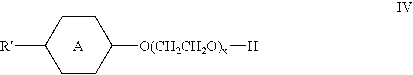
wherein R′ is (CH₃)₃C.CH₂C(CH₃)₂—,A is phenylene, and X is 9-10 or R′ (CH₃)₃C.C₂C(CH₃)₂—,
A is cyclohexylene, and X is 9-10,
or a combination thereof.

20. A method to stabilize a composition comprising Adenovirus, comprising adding to the composition a stabilizing-effective amount of a non-ionic detergent of Formula I
R—O(CH₂CH₂O)_{X}—H I
wherein R is (CH₃)(CH₂)_{Y}—, and
wherein X is 23 and Y is 12, or X is 20 and Y is 12,
or Formula II 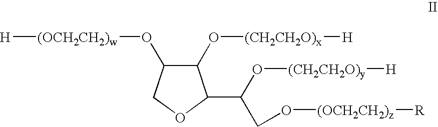
wherein R is R═C₁₁H₂₃CO₂—,
or a combination thereof.

21. A method for stabilizing a composition comprising an airborne virus, comprising adding to the composition a stabilizing-effective amount of a non-Ionic detergent of Formula I
R—O(CH₂CH₂O)_{X}—H I
wherein X is 4-30, and R is a linear or branched alkyl of 10-70 carbon atoms,
or Formula II 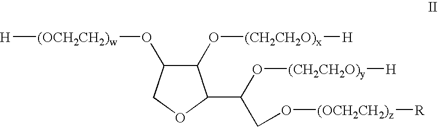
wherein R is a —CO₂R′ having 10 to 70 carbons, and W+X+Y+Z=20, wherein R′ is as defined above for R,
or Formula III 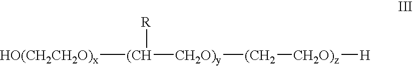
wherein R is as above, and X is 5-100, Y is 25-75 and Z is 50-100,
or Formula IV. 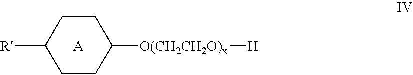
wherein X is 5-15, ring A is phenylene or cyclohexylene, and R′ is R as above,
or a combination thereof, in a concentration ranging from about 0.005% to about 0.1% (vol/vol).

22. The method of claim 21, wherein said alkyl is substituted by one or more of carboxy, carbamide, halogen, hydroxy or amine, or by 1-3 rings, which can be aromatic or cycloalkyl or can also be heterocyclic.

23. The method of claim 22, wherein said 1-3 rings can be substituted by one or more of alkyl, hydroxy, amine, halogen, nitro, sulfoxy, carboxy or carbamide.

24. A method for stabilizing a composition comprising an airborne virus, comprising adding to the composition a stabilizing-effective amount of a non-ionic detergent of Formula I.
R—O(CH₂CH₂O)_{X—H} I
wherein X is 4-30, and R is (CH₃)(CH₂)_{Y}—, wherein Y is 10-15,
or Formula III 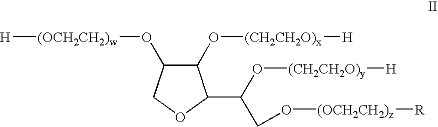
wherein R is CₐH₍₂ₐ₊₁₎CO₂—, a is 10 to 70,
or Formula III 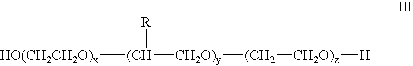
wherein R is CH₃, X is 55 Y is 29 and Z is 55, or R is CH₃, X is 98, Y is 67 and Z is 98,
or Formula IV, 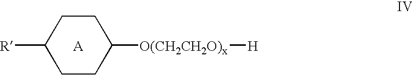
wherein R′ is (CH₃)₃C.CH₂C(CH₃)₂—, A is phenylene, and X is 9-10, or R′ (CH₃)₃C.CH₂C(CH₃)₂—,
A is cyclohexylene, and X is 9-10,
or a combination thereof.

25. A method to stabilize a composition comprising airborne virus, comprising adding to the composition a stabilizing-effective amount of a non-ionic detergent of Formula I
R—O(CH₂CH₂O)_{X}—H I
wherein R is (CH₃(CH₂)_{Y}—, and wherein X is 23 and Y is 12, or X is 20 and Y is 12,
or Formula II 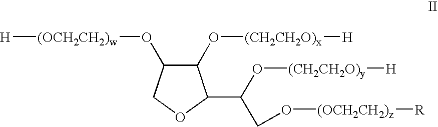
wherein R is R═C₁₁H₂₃CO₂—,
or a combination thereof.

26. A method for stabilizing a composition comprising an airborne virus to reduce loss of virus amount or activity comprising adding to the composition a stabilizing-effective amount of a non-ionic detergent of Formula I
R—O(CH₂CH₂O)_{X}—H I
wherein
X is 4-30, and R is a linear or branched alkyl of 10-70 carbon atoms,
or Formula II 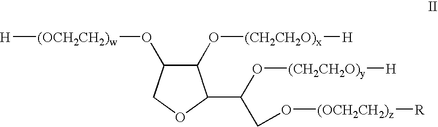
wherein R is —CO₂R′ having 10 to 70 carbons, and W+X+Y+Z=20, wherein R′ is as above for R,
or Formula III 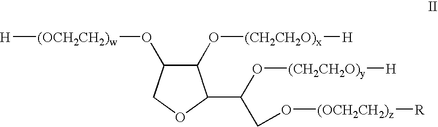
wherein R is as above, and X is 5-100, Y is 25-75 and Z is 50-100,
or Formula IV 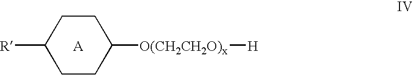
wherein X is 5-15 ring A is phenylene or cyclohexylene, and R′ is R as above, or a combination thereof, wherein the loss of virus amount or activity is less than about 30% over a given period of time at about 2-8° C., room temperature, 37° C., −20° C. or −70° C. compared to the loss when said non-ionic detergent is not present.

27. The method of claim 26 wherein said loss is less than about 10%

28. The method of claim 27 wherein said loss is less than about 5%.

29. The method of claim 28 wherein said loss is less than about 2%.

30. A pharmaceutical composition comprising an airborne virus a stabilizing-effective amount of a non-ionic detergent of Formula I,
R—O(CH₂ CH₂O)_{X}—H I
wherein
X is 4-30 and R is a linear or branched alkyl of 10-70 carbon atoms,
or Formula II 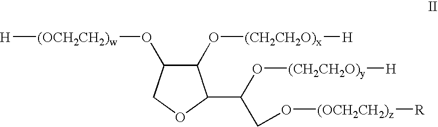
wherein R IS —CO₂R′ having 10 to 70 carbons, and W+X+Y+Z=20, wherein R′ is as above for R,
or Formula III 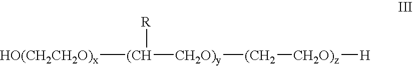
wherein R is as above, and X is 5-100, Y is 25-75 and Z is 50-100,
or Formula IV 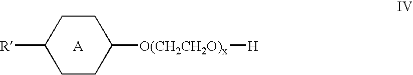
wherein X is 5-15 ring A is phenylene or cyclohexylene, and R′ is R as above,
or a combination thereof and at least one pharmaceutically acceptable carrier, salt or excipient.

31. The composition of claim 30, wherein said airborne virus is an Adenovirus.

32. The composition of claim 30, wherein the non-ionic detergent is a Brij detergent, a polysorbate (Tween) detergent, a pluronic molecule, or a Triton-like molecule.

33. The composition of claim 30, wherein, the non-ionic detergent is present at a concentration ranging from about 0.005% to about 0.1% (vol/vol).

34. The composition of claim 33, wherein the concentration ranges from about 0.05% to about 0.08% (vol/vol).

35. The composition of claim 34, wherein the concentration is about 0.05% (vol/vol).

36. A pharmaceutical composition comprising an Adenovirus, Tween 20 at a concentration of about 0.05% (vol/vol), and a pharmaceutically acceptable carrier comprising 2 mM MgCl₂ , 2% sucrose (wt/vol) and water.

37. A pharmaceutical composition, comprising
a) an Adenovirus,
b) a stabilizing-effective amount of a non-ionic detergent of Formula I
R—O(CH₂CH₂O)_{X}—H I
wherein
X is 4-30, and R is a linear or branched alkyl of 10-70 carbon atoms,
or Formula II 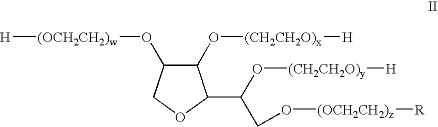
wherein R is —CO₂R′ having 10 to 70 carbons, and W+X+Y+Z=20, wherein R′ is as
above for R,
or Formula III 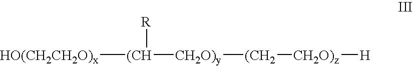
wherein R is as above, and X is 5-100, Y is 25-75 and Z is 50-100,
or Formula IV 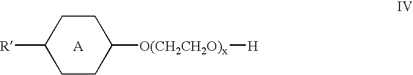
wherein X is 5-15 ring A is phenylene or cyclohexylene, and R′ is R as above,
or a combination thereof, and
c) at least one pharmaceutically acceptable carrier.

38. The composition of claim 37, wherein the Adenovirus is a recombinant Adenovirus.

39. The composition of claim 37, wherein the Adenovirus is a recombinant Adenovirus suitable for gene therapy.

40. The composition of claim 37, wherein the detergent is present at a concentration ranging from 0.005% to 0.1% (vol/vol).

41. The method of claim 15, wherein said detergent is present at a concentration ranging from 0.005% to 0.1% (vol/vol).

42. The method of claim 15, wherein the Adenovirus is a recombinant Adenovirus.

43. The method of claim 42, wherein the recombinant Adenovirus is suitable for gene therapy.

44. A pharmaceutical composition, comprising a stabilized Adenovirus composition made by the method of claim 1 and at least one pharmaceutically acceptable carrier.

45. A pharmaceutical composition, comprising a stabilized airborne virus composition made by the method of claim 1 and at least one pharmaceutically acceptable carrier.

46. The method of claim 25, wherein the detergent is polysorbate 20 (Tween 20).

47. The method of claim 7, wherein the detergent is in a concentration of 0.005% to 0.1% (vol/vol).

48. A pharmaceutical composition, comprising
a) an airborne virus,
b) a stabilizing-effective amount of a non-ionic detergent of according to Formula I
R—O(CH₂CH₂O)_{X}—H I
wherein
X is 4-30, and R is a linear or branched alkyl of 10-70 carbon atoms, or Formula II 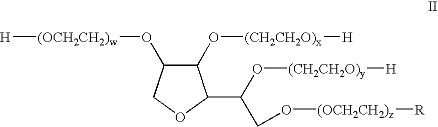
wherein R is —CO₂R′ having 10 to 70 carbons, and W+X+Y+Z=20, wherein R′ is as above for R,
or Formula III 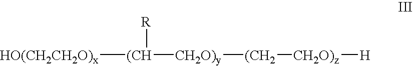
wherein R is as above, and X is 5-100, Y is 25-75 and Z is 50-100,
or Formula IV 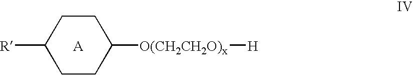
wherein X is 5-15 ring A is phenylene or cyclohexylene, and R′ is R as above,
or a combination thereof; and
c) at least one pharmaceutically acceptable carrier.

49. The composition of claim 48, wherein the detergent is in a concentration of 0.005% to 0.1% (vol/vol).

50. The method of claim 1, wherein said alkyl is substituted by one or more of carboxy, carbamide, halogen, hydroxy or amine, or by 1-3 rings, which can be aromatic or cycloalkyl or can also be heterocyclic.

51. The method of claim 49, wherein said 1-3 rings can be substituted by one or more of alkyl, hydroxy, amine, halogen, nitro, sulfoxy, carboxy or carbamide.
